(19) United States
(12) Patent Application Publication
(10) Pub. No.: US 2014/0088702 A1
(43) Pub. Date: Mar. 27, 2014

(54) TISSUE EXPANDER WITH SELF-HEALING ANTERIOR SIDE

(71) Applicant: Allergan, Inc., Irvine, CA (US)

(72) Inventors: Nicholas J. Manesis, Escondido, CA (US); Nikhil S. Trilokekar, Goleta, CA (US); Dustin Leslie, Santa Barbara, CA (US); Kaustubh S. Chitre, Goleta, CA (US)

(73) Assignee: Allergan, Inc., Irvine, CA (US)

(21) Appl. No.: 14/094,618

(22) Filed: Dec. 2, 2013

Related U.S. Application Data
(63) Continuation of application No. 13/291,695, filed on Nov. 8, 2011.
(60) Provisional application No. 61/411,273, filed on Nov. 8, 2010.

(51) Int. Cl. A61M 29/02; A61F 2/12

(52) U.S. Cl. CPC A61M 29/02; A61F 2/12
USPC 623/8; 606/192; 156/146

(57) ABSTRACT

A tissue expander includes an elastomeric shell having an anterior inside surface and a posterior inside surface with a self-healing layer abutting only a portion of the anterior inside surface. The layer is spaced apart from the posterior inside surface while facilitating folding of the shell prior to insertion into a tissue pocket and also enabling inflation of the shell subsequent to insertion by a fluid injected between the self healing layer and posterior surface.

The present application is a continuation application of pending U.S. patent application Ser. No. 13/291,695, filed on Nov. 8, 2011, which claims priority to U.S. Provisional Patent Application No. 61/411,273, filed Nov. 8, 2010, the entire disclosure of each of these applications being incorporated by this reference herein.

The present invention generally relates to implantable devices which can be infused with fluid in order to cause tissue expansion.

In the case of a mastectomy, much of the mammary glands and surrounding tissue is removed, which creates a void, which can thereafter be filled with an implantable prosthesis.

Often before the implantation of a permanent prosthesis, it is desirable to utilize what is referred to as a tissue expander, in order to enlarge, or grow, a skin flap over the cavity for accommodation of the permanent prosthesis.

After implantation, the tissue expander is gradually enlarged by the infusion of fluid. This may be accomplished with an infusion needle. After gradual inflation over periods of weeks or months, the skin and subcutaneous tissue expands in order to accommodate a permanent prosthesis.

Since a tissue expander shell may leak if punctured by a needle, it is common practice to infuse fluid at a location that is remote from the tissue expander shell. A tissue expander system thus generally includes a remote needle penetrable septum which is inserted through a remote port connected to the tissue expander by means of a conduit. Known tissue expansion systems often require that the septum and the conduit that connects the septum to the tissue expander prosthesis be implanted with the tissue expander prosthesis. The surgery for implanting a tissue expansion system normally includes an incision or incisions through which the implant and conduit and septum are directed. It can be appreciated that a tissue expander with a remote septum requires a greater amount of surgery to implant than a tissue expander without a septum.

Bark, et al. describes a self-sealing tissue expander which includes a closed flexible shell that defines an internal chamber having no fluid entry port and is noncommunicable with any septum or conduit. The shell is puncturable with a needle and is self-sealing. Fluid infusion is accomplished directly through the shell.

A self-healing tissue expander is desirable, however, the thickness of self-healing expandable walls can prevent compact folding of the expander prior to insertion into a tissue pocket.

Accordingly, it is desirable to provide an improved tissue expander for preparing a breast for a more permanent mammary prosthesis, or a prosthesis in another area of the body.

### SUMMARY

A tissue expander, hereinafter sometimes referred to as an implant, is provided. The expander generally includes an elastomeric shell having an anterior inside surface and a posterior inside surface, and a fluid fillable, expandable chamber therebetween.

The expander further comprises a self-sealing layer, hereinafter sometimes referred to as “self-healing layer”. The self-healing layer abuts at least a portion of the anterior inside surface. In one aspect of the invention, the self-healing layer extends only as far as the posterior inside surface. In another aspect of the invention, the self-healing layer does not extend as far as the posterior inside surface and includes boundaries which are spaced apart from the posterior inside surface. The entire shell is foldable which facilitates surgical introduction of the implant insertion into a tissue pocket and further enables inflation of the shell subsequent to insertion by fluid injected between the self-healing layer and the posterior surface.

A self-healing layer may be formed from a suitable silicone gel. The self-healing layer may be adhered to the anterior inside surface with an adhesive, for example, a silicone-based adhesive.

In some embodiments, the self-healing layer comprises a silicone gel which is located between the anterior inside surface and a partition. The partition forms a pocket, which is fixed to the anterior inside surface to form a gel pocket. The partition may be fixed to the anterior inside surface by an adhesive and a silicone gel may be disposed in the pocket.

In another aspect, a folded tissue expander is provided. The folded tissue expander is formed of an elastomeric shell with an anterior inside surface and a posterior inside surface along with a self-healing layer adhered to at least a portion of the anterior inside surface. The self-healing layer may abut the posterior inside surface or may be spaced apart therefrom. In the folded configuration, the elastomeric shell may be inserted into a surgically-created breast pocket and then expanded by injection of fluid between the posterior inside surface and the self-healing layer. After infusing the cavity with saline, the needle is withdrawn without the occurrence of leaking.

### BRIEF DESCRIPTION OF THE DRAWINGS

The advantages and features of the present invention may be better understood and/or appreciated by the following description when considered in conjunction with the accompanying drawings in which:

FIG. 1 is a cross-sectional view of a tissue expander in accordance with an embodiment of present invention, generally showing an elastomeric shell having a self-healing layer abutting only a portion of an anterior inside surface.

FIG. 2 is an alternative configuration of a tissue expander in accordance with the present invention similar to that shown in FIG. 1 wherein the self-healing layer includes a partition fixed along a perimeter thereof to an anterior surface to form a gel pocket with a silicone disposed therein; and

FIG. 3 is a folded configuration of a tissue expander in accordance with the present invention illustrating a compact folded expander facilitated by a use of a self-healing layer disposed only on one anterior surface of the tissue expander.

### DETAILED DESCRIPTION

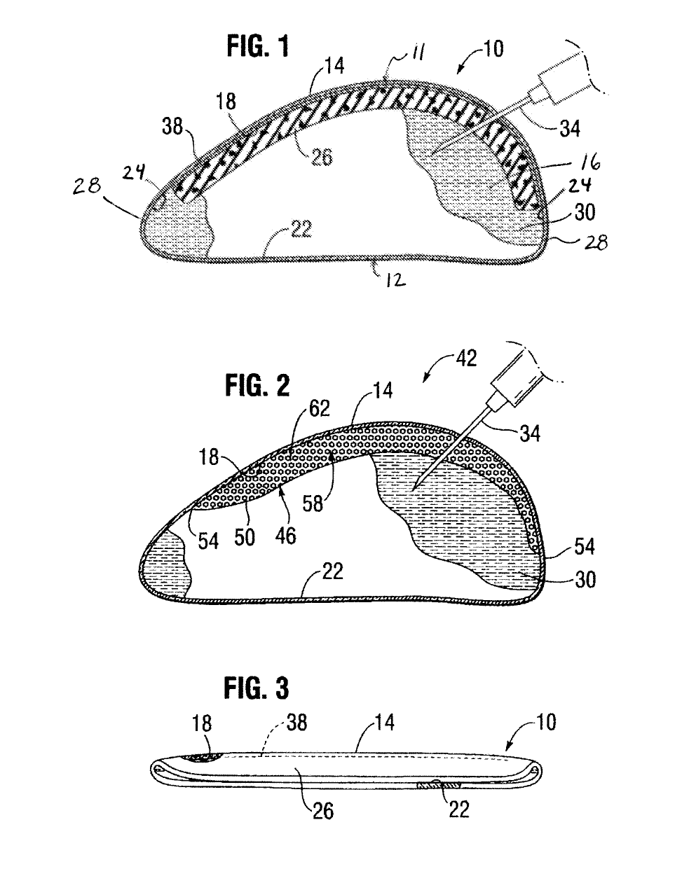

With reference to FIG. 1, there is shown a tissue expander 10 in accordance with an embodiment of the present invention. The expander 10 is generally formed in the shape of a breast implant, with a rounded anterior portion 11 and a generally planar posterior portion 12, forming a shell 14 of the expander 10. It can be appreciated by those of skill in the art that the expander can take on other shapes as well, depending upon the part of the body in which the expander is to be placed, and the specific needs of the patient requiring the expander.

In the shown embodiment, generally defined between the anterior portion 11 and the posterior portion 12 is a fluid-fillable cavity 16 which, when infused with fluid, for example saline or other biologically inert fluid, enlarges the volume of expander. The expander 10, once positioned in a breast pocket created by a mastectomy, for example, can therefore be gradually inflated to allow tissue such as skin, to grow slowly, for example, to accommodate the tissue expander 10 without causing unnecessary trauma to the patient's body. Once the breast pocket is appropriately sized, the tissue expander 10 is removed from the breast and a more permanent breast implant may be surgically implanted into the pocket that is left after removal of the expander 10.

The anterior and posterior portions 11, 12 forming the shell 14 of the expander 10, may form a single unitary construction which has been formed by traditional dip molding techniques, on a mold surface, for example, a mandrel, as is well known in the art. The shell 14 may be formed of any suitable elastomeric material, for example, a silicone elastomer, for example, a silicone elastomer manufactured under the tradename PN-3206-1, available from Nusil, Inc., or other suitable, biocompatible elastomer.

The elastomeric shell 14 includes an anterior inside surface 18 and a posterior inside surface 22.

As shown in FIG. 1, a self-healing layer 26 covers or abuts only a portion of the anterior inside surface 18. As also shown in FIG. 1, the self-healing layer 26 may be disposed in a spaced apart relationship with the posterior inside surface 22. For example, as in the shown embodiment, the self-healing layer 26 does not extend as far as to contact the curved surface region 28 that generally defines the widest perimeter of the expander located between the dome-shaped anterior portion and the generally planar posterior portion forming the shell. A perimeter portion 24 of the anterior inside surface 18 is left uncovered by the self-healing layer.

The structure of the self healing layer 26 may be selected in order to enable healing of any hole created by a infusion needle, for example, a 21 gauge or finer diameter hypodermic needle 34. The self-healing layer prevents any leakage of saline solution when the needle 34 is removed from the expander 10.

Turning briefly to FIG. 3, the structure of the presently described tissue expanders may significantly facilitate folding thereof prior to insertion into a breast pocket (not shown). Further, the structure may facilitate inflation of the shell 14, as shown in FIG. 1, with a fluid 30, for example, a saline solution, by a hypodermic needle 34.

The self-healing layer 26 may be cast separately from a soft silicone, for example, any suitable silicone gel, for example, a clear, tacky silicone gel, for example, a medium penetration soft silicone gel known to those of skill in the silicone gel art, such as, but not limited to MED 6350, available from Nusil Technologies, Inc. The layer 26 may be cast in a sheet form and thereafter cut and attached to the anterior inner surface 18. Attachment to the inner surface 18 may be accomplished using a silicone adhesive 38. This may be done after molding of the shell 14 by turning the molded shell inside-out, applying the adhesive 38 and then applying the layer 26.

An alternative embodiment tissue expander 42 is shown in FIG. 2 with common numerical references referring to identical or substantially equivalent elements illustrated in FIG. 1 in the description of the tissue expander 10.

Tissue expander 42 includes a self-healing layer 46 which includes a separate partition 50 fixed along a perimeter 54 thereof to the anterior inside surface 18 of the shell 14 to form a gel pocket (shown filled with a gel 62 in FIG. 2). Any suitable cohesive gel 62 may be utilized and the pocket 58 is of sufficient thickness, empirically determined, to enable self-healing after penetration by a 21G or smaller hypodermic needle 34 as shown in FIG. 2.

The tissue expander 42 may further include a needle guard (not shown) forming or covering a posterior side of the expander 42. Such a needle guard can be provided to prevent a needle from undesirably puncturing entirely through the expander 42.

### EXAMPLE 1

### Method Of Making A Self-Healing Tissue Expander

A silicone gel dispersion, PN-3206-1 (Nusil Technologies) was used for constructing a flexible silicone shell. A different soft silicone gel, Nusil MED 6350 was used to form the self healing layer shown such as shown in FIG. 1.

The shell is created by mandrel dipping process known to those of skill in the art.

Soft silicone MED 6350 was casted separately in the form of gel sheet and cured at about 150 C for about 30 min.

When fully cured, a patch of appropriate dimension was cut from the silicone gel sheet and is then attached on the inner surface of anterior side of the shell, the cut gel sheet forming the self-healing layer of a tissue expander such as shown in FIG. 1.

### EXAMPLE 2

### Method Of Making A Self-Healing Tissue Expander

A silicone gel dispersion, PN-3206-1 (Nusil Technologies) was used for constructing the shell.

A portion of an identical shell was cut and secured to the inside surface of the shell using a silicone adhesive to form a pocket. The pocket was filled with a cohesive silicone gel and the pocket is sealed around the cohesive silicone gel using silicone adhesive. The pocket and cohesive gel form a self-healing layer of the tissue expander such as shown in FIG. 2.

Although there has been hereinabove described a specific tissue expander with self-healing anterior side in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element, which is not specifically disclosed herein. Accordingly, any and all modifications, variations or equivalent arrangements which may occur to those skilled in the art, should be considered to be within the scope of the present invention as defined in the appended claims.

What is claimed is:

1. An inflatable tissue expander for an implantation in a breast, the expander comprising:
an elastomeric shell having an anterior inside surface including a perimeter portion, and a posterior inside surface;
a partition fixed to the anterior inside surface and forming a pocket between the partition and the anterior inside surface; and
a self-healing layer comprising a gel, located in the pocket;
the perimeter portion of the anterior inside surface being left uncovered by the self-healing layer.

2. The expander according to claim 1 wherein said self-healing layer is formed from a silicone gel.

3. The expander according to claim 1 in a rolled configuration.

4. The expander according to claim 1 wherein the shell, the partition and the self-healing layer are all comprised of silicone.

5. An inflatable shell for a breast tissue expander, the shell comprising:
an anterior inside surface including a perimeter portion, and a posterior inside surface;
a partition fixed to the anterior inside surface and forming a pocket between the partition and the anterior inside surface; and
a self-healing layer comprising a gel, located in the pocket;
the perimeter portion of the anterior inside surface being left uncovered by the self-healing layer.

6. The shell according to claim 5 wherein said self-healing layer is formed from a silicone gel.

7. The shell according to claim 5 in a rolled configuration.

8. A method of making a tissue expander for a breast, the method comprising:
providing a shell having an anterior inside surface and a posterior inside surface defining a fluid-fillable cavity therebetween;
fixing a partition to the anterior inside surface of the shell to form a pocket between the anterior inside surface and the partition;
filling the pocket with a cohesive gel; and
sealing the pocket around the cohesive gel using an adhesive, to form a layer on the shell that is self-sealing to needle puncture.

9. The method according to claim 8 wherein the cohesive gel is a silicone gel.

10. The method of claim 8 wherein the shell, the partition, and the cohesive gel are all comprised of silicone.

11. The method of claim 8 wherein the shell, the partition, the cohesive gel, and the adhesive are all comprised of silicone.
